(19) United States
(12) Patent Application Publication
(10) Pub. No.: US 2012/0157791 A1
(43) Pub. Date: Jun. 21, 2012

(54) ADAPTIVE TIME DOMAIN FILTERING FOR IMPROVED BLOOD PRESSURE ESTIMATION

(75) Inventor: Lawrence T. Hersh, Milwaukee, WI (US)

(73) Assignee: GENERAL ELECTRIC COMPANY, Schenectady, NY (US)

(21) Appl. No.: 12/970,103

(22) Filed: Dec. 16, 2010

(51) Int. Cl. A61B 5/00

(52) U.S. Cl. 600/301

(57) ABSTRACT

A system and method for processing a cuff pressure waveform to determine the blood pressure of a patient. A heart rate monitor acquires the patient's heart rate. Based upon the acquired heart rate, the system selects filtering parameters for processing the cuff pressure waveform received from the patient. The filtering parameters include a high pass cutoff frequency and a low pass cutoff frequency that are determined based upon the heart rate of the patient. The low pass cutoff frequency is based upon a harmonic frequency of the heart rate while the high pass cutoff frequency is based upon the fundamental frequency of the heart rate. The high pass and low pass cutoff frequencies are used to select filtering coefficients. The high pass and low pass cutoff frequencies are selected based upon the heart rate of the patient such that the filtering adapts based on the heart rate of the patient.

### BACKGROUND OF THE INVENTION

The present disclosure generally relates to the field of non-invasive blood pressure monitoring. More specifically, the present disclosure relates to a method and system for filtering a cuff pressure waveform from a patient in the time domain using filter parameters based on the determined heart rate of the patient for the improved processing of the cuff pressure waveform.

The human heart periodically contracts to force blood through the arteries. As a result of this pumping action, pressure pulses or oscillations exist in these arteries and cause them to cyclically change volume. The minimum pressure during each cycle is known as the diastolic pressure and the maximum pressure during each cycle is known as the systolic pressure. A further pressure value, known as the “mean arterial pressure” (MAP) represents a time-weighted average of the measured blood pressure over each cycle.

While many techniques are available for the determination of the diastolic, systolic, and mean arterial pressures of a patient, one such method typically used in non-invasive blood pressure monitoring is referred to as the oscillometric technique. This method of measuring blood pressure involves applying an inflatable cuff around an extremity of a patient's body, such as the patient's upper arm. The cuff is then inflated to a pressure above the patient's systolic pressure and then incrementally reduced in a series of small pressure steps. A pressure sensor pneumatically connected to the cuff measures the cuff pressure throughout the deflation process. The sensitivity of the sensor is such that it is capable of measuring the pressure fluctuations occurring within the cuff due to blood flowing through the patient's arteries. With each beat, blood flow causes small changes in the artery volume which are transferred to the inflated cuff, further causing slight pressure variations within the cuff which are then detected by the pressure sensor. The pressure sensor produces an electrical signal representing the cuff pressure level combined with a series of small periodic pressure variations associated with the beats of a patient's heart for each pressure step during the deflation process. It has been found that these variations, called “complexes” or “oscillations,” have a peak-to-peak amplitude which is minimal for applied cuff pressures above the systolic pressure.

As the cuff pressure is decreased, the oscillation size begins to monotonically grow and eventually reaches a maximum amplitude. After the oscillation size reaches the maximum amplitude, the oscillation size decreases monotonically as the cuff pressure continues to decrease. Oscillometric data such as this is often described as having a “bell curve” appearance. Indeed, a best-fit curve, or envelope, may be calculated representing the amplitude of the measured oscillometric pulses. Physiologically, the cuff pressure at the maximum oscillation amplitude value approximates the MAP. In addition, complex amplitudes at cuff pressures equivalent to the systolic and diastolic pressures have a fixed relationship to this maximum oscillation amplitude value. Thus, the oscillometric method is based upon measurements of detected oscillation amplitudes at various cuff pressures.

Blood pressure measuring devices operating according to the oscillometric method detect the amplitude of the pressure oscillations at various applied cuff pressure levels. The amplitudes of these oscillations, as well as the applied cuff pressure, are stored together as the device automatically changes the cuff pressures through a predetermined pressure pattern. These oscillation amplitudes define an oscillometric “envelope” and are evaluated to find the maximum value and its related cuff pressure, which is approximately equal to MAP. The cuff pressure below the MAP value which produces an oscillation amplitude having a certain fixed relationship to the maximum value is designated as the diastolic pressure, and, likewise, the cuff pressures above the MAP value which results in complexes having an amplitude with a certain fixed relationship to that maximum value is designated as the systolic pressure. The relationships of oscillation amplitude at systolic and diastolic pressures, respectively, to the maximum value at MAP are empirically derived ratios depending on the preferences of those of ordinary skill in the art. Generally, these ratios are designated in the range of 40%-80% of the amplitude at MAP.

One way to determine oscillation magnitudes is to computationally fit a curve to the recorded oscillation amplitudes and corresponding cuff pressure levels. The fitted curve may then be used to compute an approximation of the MAP, systolic and diastolic data points. An estimate of MAP is taken as the cuff pressure level with the maximum oscillation. One possible estimate of MAP may therefore be determined by finding the point on the fitted curve where the first derivative equals zero. From this maximum oscillation value data point, the amplitudes of the oscillations at the systolic and diastolic pressures may be computed by taking a percentage of the oscillation amplitude at MAP. In this manner, the systolic data point and the diastolic data point along the fitted curve may each be computed and therefore their respective pressures may also be estimated. This curve fitting technique has the advantage of filtering or smoothing the raw oscillometric data. However, in some circumstances it has been found that additional filtering techniques used to build and process the oscillometric envelope could improve the accuracy of the determination of the blood pressure values.

The reliability and repeatability of blood pressure computations hinges on the ability to accurately determine the oscillation amplitude. However, the determination of the oscillation amplitudes is susceptible to artifact contamination. Since the oscillometric method is dependent upon detecting tiny fluctuations in measured cuff pressure, outside forces affecting this cuff pressure may produce artifacts that in some cases may completely mask or otherwise render the oscillometric data useless. One such source of artifacts is from voluntary or involuntary motion by the patient. Involuntary movements, such as the patient shivering, may produce high frequency artifacts in the oscillometric data. Voluntary motion artifacts, such as those caused by the patient moving his or her arm, hand, or torso, may produce low frequency artifacts.

Presently available systems may be able to determine whether or not collected oscillometric data has been corrupted with artifact; however, some current filtering techniques are carried out in the frequency domain and require the use of a fast Fourier transform (FFT) algorithm. The FFT algorithm has several restrictions that may not be desirable in all filtering cases. As an example, the FFT algorithm requires a significant amount of computational power and speed. Since computer resources may not be available in every NIBP monitoring system, the FFT algorithm can only be used in certain circumstances. Additionally, a FFT algorithm performs filtering over a specific period of time having a desired number of samples. Since the FFT algorithm requires a certain number of samples to be stored, the FFT algorithm again requires significant computational overhead. Additionally, non-invasive blood pressure systems may simply reject oscillometric data that has been designated as being corrupted by artifacts. In these instances, more oscillometric data must be collected at each pressure step until reasonably artifact free oscillometric data may be acquired. This may greatly lengthen the time for determination of a patient's blood pressure and submit the patient to increased discomfort that is associated with the inflatable cuff restricting blood flow to the associated extremity.

### SUMMARY OF THE INVENTION

A method of filtering an oscillometric signal from a patient for computing an oscillometric envelope for use in determining the blood pressure of the patient is disclosed herein. The method includes the steps of receiving a cuff pressure waveform in a processing unit. Next, the fundamental frequency and at least one harmonic frequency of the patient's heart rate are found using the heart rate of the patient, which is received from a heart rate monitor, such as an SpO₂ or ECG monitor.

A method and system of filtering the cuff pressure waveform received from a patient for use in computing an oscillometric envelope and blood pressure estimate for a patient is disclosed herein. The method and system utilizes the current heart rate of the patient to select digital filtering coefficients for processing the cuff pressure waveform received from the patient. The adaptive technique of the present disclosure selects filtering coefficients based upon the current heart rate of the patient.

Once the blood pressure cuff has been applied to the patient, the processing unit of the NIBP monitoring system inflates the pressure cuff to an initial inflation pressure. The blood pressure cuff is then deflated in a series of pressure steps. At each pressure step, the processing unit obtains information related to the heart rate of the patient. Based upon the heart rate information, the processing unit retrieves stored digital filtering coefficients. The digital filtering coefficients are selected from the stored values based upon a high pass cutoff frequency and a low pass cutoff frequency to insure that the fundamental frequency of the heart rate and the first two harmonics are included within the pass band. Although two harmonic frequencies are described as being within the scope of the present disclosure, it should be understood that additional harmonics could be utilized while operating within the scope of the present disclosure.

Once the filtering coefficients have been retrieved from a memory unit, the processing unit initializes the high and low pass digital filters and processes the cuff pressure waveform to detect oscillations. The oscillation size information and pressure level are stored within the memory of the processing unit. Since the filtering coefficients are selected based upon the heart rate of the patient, the signal from the blood pressure cuff is filtered to remove artifacts that occur outside of the pass band, which includes most of the signal energy.

Once oscillometric data has been retrieved at the pressure step, the pressure of the blood pressure cuff is reduced and the system again selects the filtering parameters based upon the current heart rate of the patient. In this manner, the system can select different filtering coefficients at each pressure step based upon the heart rate obtained at the specific pressure step. This adaptive technique insures that the energy from the oscillometric signal is detected for each pressure step since the pressure step is filtered based upon the current heart rate of the patient.

Once the oscillometric envelope has been built, the processor utilizes known techniques to determine the blood pressure for the patient. The blood pressure estimate is then output on a display and can be analyzed by medical personnel, as is known.

### BRIEF DESCRIPTION OF THE DRAWINGS

The drawings illustrate the best mode presently contemplated of carrying out the disclosure. In the drawings:

FIG. 1 depicts an embodiment of a system for the non-invasive measurement of blood pressure;

FIG. 2 is a graph depicting the oscillometric data collected from a blood pressure cuff at multiple pressure steps;

FIG. 3 is a flowchart illustrating the acquisition and operation sequence for the data used by the system of the present disclosure to determine the blood pressure of a patient;

FIG. 4 is a flowchart illustrating the steps used in the pressure waveform processing using a low pass filter and a high pass filter selected based upon the heart rate of the patient;

FIGS. 5a-5d illustrate several types of low pass filters that can be selected as part of the pressure waveform processing;

FIGS. 6a-6b illustrate several types of high pass filters that can be selected as part of the pressure waveform processing;

FIG. 7 is an alternate type of high pass filter that can be used in accordance with the disclosure;

FIG. 8 is a graph illustrating the various different cuff pressures used to determine the blood pressure of a patient and the results of the adapted filtering technique; and

FIG. 9 is a flowchart illustrating the operational sequence carried out by the processing unit of the present disclosure.

### DETAILED DESCRIPTION OF THE INVENTION

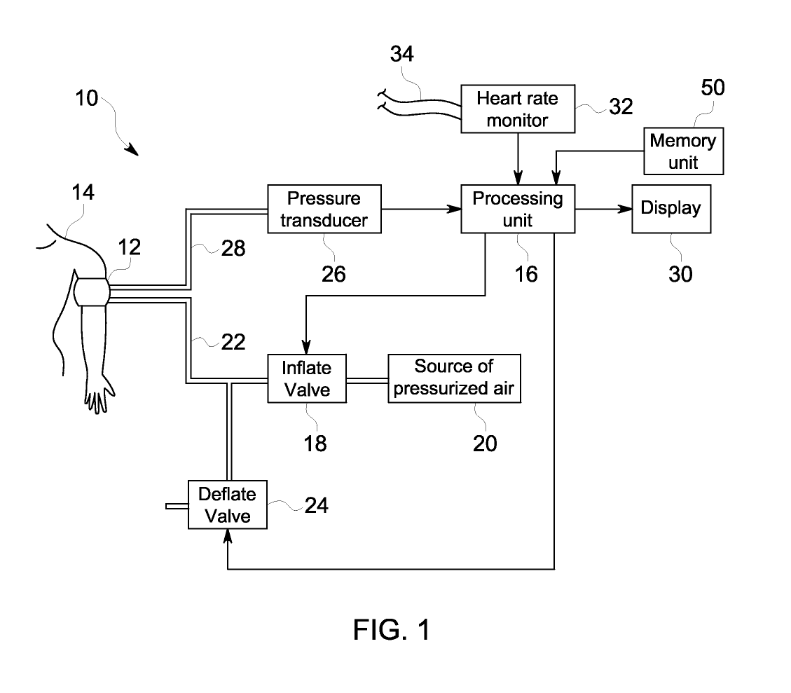

FIG. 1 depicts an embodiment of a non-invasive blood pressure (NIBP) monitoring system 10. The NIBP monitoring system 10 includes a pressure cuff 12 that is a conventional flexible, inflatable and deflatable cuff worn on the arm or other extremity of a patient 14. A processing unit 16 controls an inflate valve 18 that is disposed between a source of pressurized air 20 and a pressure conduit 22. As the inflate valve 18 is controlled to increase the pressure in the cuff 12, the cuff 12 constricts around the arm of the patient 14. Upon reaching a sufficient amount of pressure within the cuff 12, the cuff 12 fully occludes the brachial artery of the patient 14.

After the cuff 12 has been fully inflated, the processing unit 16 further controls a deflate valve 24 to begin incrementally releasing pressure from the cuff 12 back through pressure conduit 22 and out to the ambient air. During the inflation and incremental deflation of the cuff 12, a pressure transducer 26, pneumatically connected to the pressure cuff 12 by pressure conduit 28 measures the pressure within the pressure cuff 12. In an alternative embodiment, the cuff 12 is continuously deflated as opposed to incrementally deflated. In such continuously deflating embodiments, the pressure transducer 26 may measure the pressure within the cuff continuously. In a further alternative embodiment, the cuff 12 is incrementally inflated to gather the oscillometric envelope information. In yet a further alternative embodiment the cuff 12 may be incrementally deflated and inflated in a mixed but controlled pattern to gather the oscillometric envelope information.

As the pressure within the cuff 12 is controlled by the processing unit 16, the pressure transducer 26 will detect oscillometric pulses in the measured cuff pressure that are representative of the pressure fluctuations caused by the patient's blood flowing into the brachial artery with each heart beat and the resulting expansion of the artery to accommodate the additional volume of blood.

The cuff pressure data as measured by the pressure transducer 26, including the oscillometric pulses, is provided to the processing unit 16 such that the cuff pressure waveform may be processed and analyzed and a determination of the patient's blood pressure, including systolic pressure, diastolic pressure and MAP can be displayed to a clinician on a display 30.

The processing unit 16 may further receive an indication of the heart rate of the patient 14 as acquired by a heart rate monitor 32. The heart rate monitor 32 acquires the heart rate of the patient 14 using one or more of a variety of commonly used heart rate detection techniques. One heart rate detection technique that may be used would be that of electrocardiography (ECG) wherein electrical leads 34 connected to specific anatomical locations on the patient 14 monitor the propagation of the electrical activity through the patient's heart. Alternatively, the patient's heart rate may be acquired using Sp0_{2,} plethysmography, or other known techniques, including signal processing and analysis of the cuff pressure data.

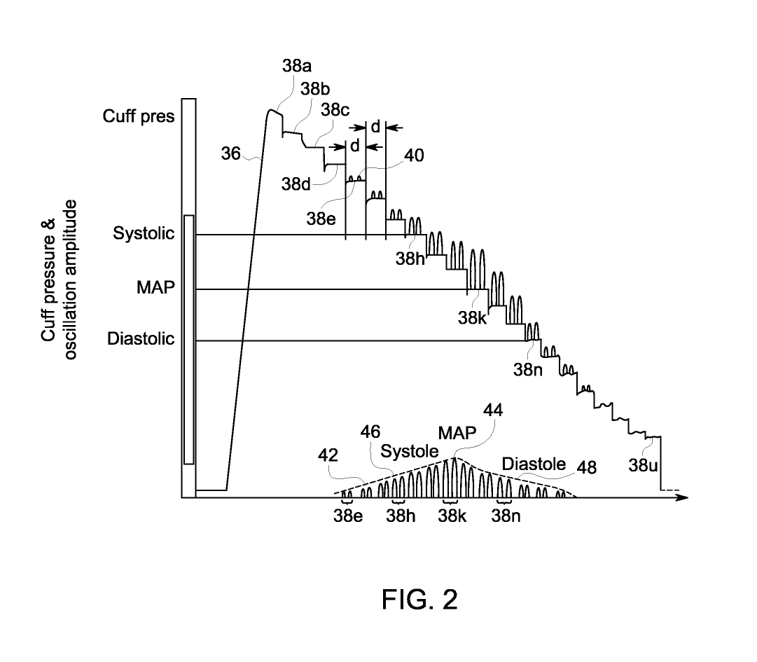

FIG. 2 is a graph depicting various pressure values that may be acquired from the NIBP monitoring system 10 depicted in FIG. 1. The cuff pressure as determined by the pressure transducer 26 is represented as cuff pressure graph 36. The cuff pressure peaks at the cuff pressure step 38a which is the cuff pressure at which the cuff 12 has been fully inflated as controlled by the processing unit 16. The processing unit 16 controls the inflation of the cuff 12 such that 38a is a pressure that is sufficiently above the systolic pressure of the patient. This may be controlled or modified by referencing previously determined values of patient blood pressure data or by reference to standard medical monitoring practices. The cuff pressure graph 36 then incrementally lowers at a series of pressure steps 38a-38u which reflect each incremental pressure reduction in the cuff 12 as controlled by the deflate valve 24. Before the cuff pressure has reached a pressure step at which the patient brachial artery is no longer completely occluded, the measured cuff pressure will show oscillometric pulses 40. The number of oscillometric pulses detected at each pressure step is controlled as a function of the heart rate of the patient and the length of time that the NIBP system collects data at each pressure step, but typically cuff pressure data is recorded at each pressure level to obtain at least two oscillometric pulses.

The cuff pressure is measured at each of the pressure step increments, including the oscillometric pulse data until the cuff pressure reaches an increment such that the oscillometric pulses are small enough to completely specify the oscillometric envelope, such as is found at pressure increment 38u. At this point, the processing unit 16 controls the deflate valve 24 to fully deflate the pressure cuff 12 and the collection of blood pressure data is complete.

FIG. 2 further depicts an oscillometric envelope 42 as calculated using the oscillometric pulse data collected from the series of incremental cuff pressure steps. The processing unit 16 isolates the oscillometric pulses at each pressure step, and creates a best-fit curve to represent the oscillometric envelope 42. The oscillometric envelope is useful in estimating systolic pressure, diastolic pressure and MAP. The MAP 44 is determined as the pressure step increment 38k that corresponds to the peak of the oscillometric envelope 42. Once the MAP has been determined, the systolic pressure 46 and diastolic pressure 48 may be identified as the pressure level values associated with particular oscillation amplitudes that are predetermined percentages of the oscillation amplitude at the MAP pressure level. In one embodiment, the systolic pressure 46 corresponds to pressure increment 38h where the oscillometric envelope amplitude is 50% that of the MAP. In another embodiment, the diastolic pressure 48 correlates to pressure increment 38n where the envelope amplitude is between 60% and 70% that of the envelope amplitude at MAP. The percentages of the MAP amplitude used to estimate the systolic pressure and the diastolic pressure are usually between 40% and 80% depending upon the specific algorithm used by the processing unit 16.

In an alternative embodiment, the amplitude of the oscillometric pulses at each pressure step are averaged to produce an oscillometric envelope data point. In some of these embodiments, techniques such as pulse matching or the elimination of the first oscillometric pulse at a pressure step may be used to improve the quality of the computed oscillometric data point. The oscillometric envelope 42 may also be created by using the average of the complex amplitudes at the pressure step as the input data points for a best-fit curve. Alternatively, data points of the oscillometric envelope 42 may be the maximum amplitude of the oscillometric pulses at each pressure step.

As can be seen, from FIG. 2, the oscillometric pulses are relatively small with respect to the overall cuff pressure and the pressure increment steps. This makes the detection of the oscillometric pulses highly susceptible to noise and other artifacts. When processing the oscillometric signal from the patient, the largest amount of physiological energy within the signal is contained at a fundamental frequency and within the first two harmonics of the heart rate of the patient. Since most of the energy is contained within the frequency band defined at a low end by the fundamental frequency and at the high end by the second harmonic frequency, time domain filtering that removes the portion of the oscillometric signal below the fundamental frequency and above the second harmonic reducese the amount of noise contained within the signal without losing any of the desirable information from the signal.

The physiological monitoring system, and method of determining blood pressure as disclosed herein, aim to provide improved processing of oscillometric pulse signals to remove artifacts. Embodiments as disclosed herein may result in producing a higher quality oscillometric pulse signal when the desired physiological signal and the artifact have specific frequency content properties; this leads to increased accuracy in constructing the oscillometric envelope and computation of the patient blood pressure estimates. FIG. 2 demonstrates an example of acquisition of the oscillometric signals using step deflation; however, other techniques of obtaining the oscillometric signals, such as by continuous deflation or step inflation, are possible, and the description given here is not meant to limit the usefulness of embodiments as disclosed below with respect to step deflation.

Referring back to FIG. 1, when calculating an automated NIBP measurement in the processing unit 16, it is important not to let artifacts cause inaccuracies in the reported blood pressure estimates. In accordance with the present disclosure, the processing unit 16 filters the cuff pressure waveform obtained from the pressure transducer 26 before the waveform is analyzed within the processing unit 16 for information that is used to determine the blood pressure estimates. In accordance with the present disclosure, the processing unit 16 utilizes adaptive time domain filtering on the cuff pressure waveform from the pressure transducer 26. The adaptive time domain filtering is accomplished by creating a series of NIBP filtering coefficients that are stored within a memory unit 50. The coefficients stored in the memory unit 50 are determined by designating a series of filters that can be used with the NIBP monitoring system 10. The filter coefficients stored in the memory unit 50 are retrieved by the processing unit 16 depending upon a parameter from the patient, such as the heart rate.

As previously described, the heart rate monitor 32 provides an indication to the processing unit 16 of the heart rate of the patient. The heart rate monitor 32 can be either an ECG or SpO₂ monitor. Alternatively, the heart rate monitor 32 could be any type of monitor that returns information to the processing unit 16 to indicate the heart rate of the patient.

In the present disclosure, the heart rate monitor 32 provides a signal to the processing unit that indicates the heart rate of the patient. However, the heart rate monitor could simply provide the signal from the patient and the processing unit 16 could be programmed to determine the heart rate of the patient. In such an embodiment, the processing capabilities would be removed from the heart rate monitor 32 and incorporated into the processing unit 16. In either case, the processing unit 16 obtains an indication of the heart rate of the patient through the heart rate monitor 32.

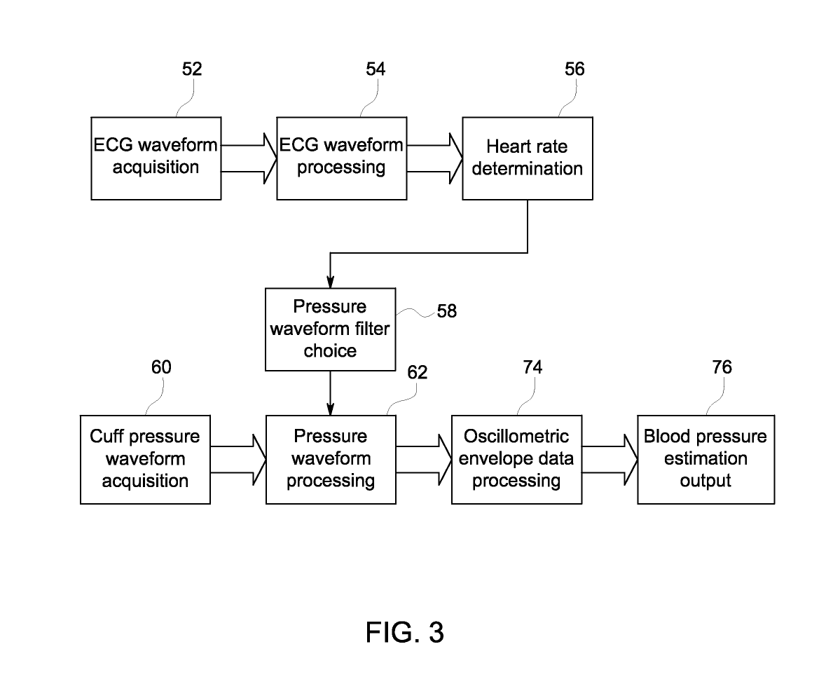

FIG. 3 generally describes the operation of the processing unit 16 in determining the blood pressure of the patient. In step 52, the NIBP monitoring system initially acquires ECG waveform information from an ECG monitor. In the embodiment shown in FIG. 3, the heart rate monitor is an ECG waveform acquisition device. However, it should be understood that similar steps would be carried out if the heart rate monitor were an SpO₂ monitoring system.

Once the ECG waveform has been acquired from the patient, the heart rate monitor conducts ECG waveform processing in step 54 to generate a heart rate determination in step 56. As previously described, the heart rate is determined within the heart rate monitor in the embodiment shown in the present application but could be calculated in the processing unit in an alternate embodiment.

Once the heart rate determination has been made in step 56, the system proceeds to step 58 in which the system selects a waveform filter based upon the heart rate from the patient. The selection made in step 58 includes selecting a coefficient set for both a desired high pass cutoff frequency and a low pass cutoff frequency. The high pass and low pass cutoff frequencies are specifically selected based upon the heart rate of the patient. Specifically, the high pass and low pass cutoff frequencies are selected based upon the harmonic content that is needed in order to keep the most relevant physiological information from the signal from the blood pressure cuff while discarding motion artifacts that arise from external interferences such as from the muscle contractions of the patient or a surgeon leaning on the blood pressure cuff during a procedure which requires vigorous physical manipulation of the patient.

As an illustrative example, if the heart rate determined in step 54 for the patient is 60 bpm, the fundamental frequency of the heart rate is 1 Hz while the first and second harmonics are 2 Hz and 3 Hz, respectively. Since most of the physiological information is contained within the fundamental frequency and the first two harmonics, the pressure waveform filter selected in step 58 is based upon the fundamental frequency and the first two harmonics. In the illustrative example in which the heart rate is 60 bpm, the low pass cutoff frequency would be 3 Hz to include the first two harmonics and the high pass cutoff frequency would be 1 Hz to insure that the fundamental frequency is included.

As another illustrative example, if the heart rate were determined to be 120 bpm, the fundamental frequency and first two harmonics are 2 Hz, 4 Hz and 6 Hz, respectively. In such an embodiment, the low pass cutoff frequency would be selected to 6 Hz while the high pass cutoff frequency would be selected 2 Hz to insure that the fundamental frequency is included in the filtering set.

In step 58, the processing unit 16 of FIG. 1 selects which type of waveform filter will be best to filter the signals based upon the heart rate from the heart rate monitor 32. Based upon this selection, the processing unit 16 retrieves a set of digital filter coefficients from the memory unit based upon the selected high pass and low pass cutoff frequencies. As previously described, the high pass and low pass cutoff frequencies are based upon the heart rate from the patient and the desired number of harmonics used by the filtering technique. In an alternate embodiment, more than two harmonics could be utilized. As an example, if three harmonics were used and the patient's heart rate was 120 bpm, the low pass cutoff frequency would be 8 Hz, rather than the 6 Hz low pass cutoff frequency described above when only two harmonics are used.

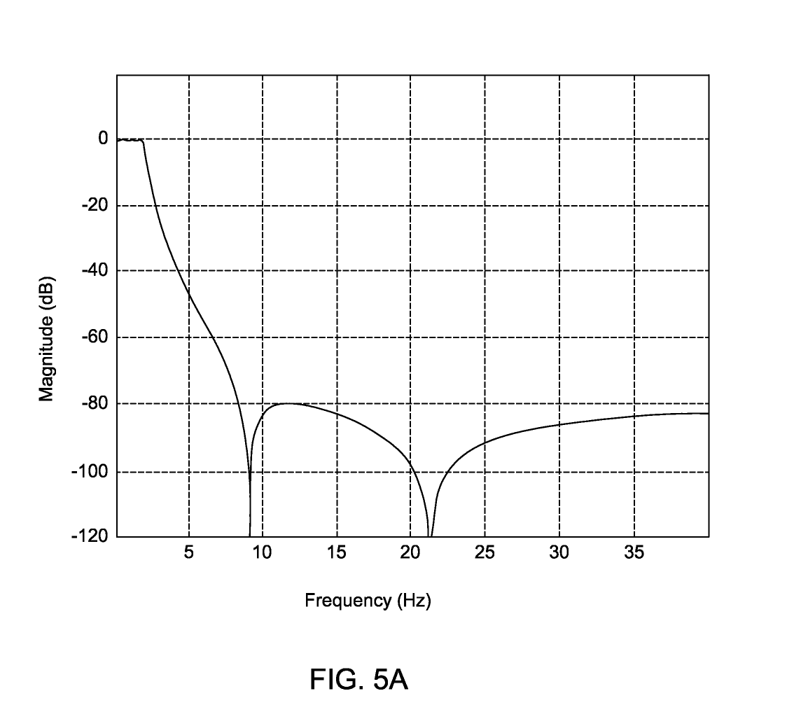

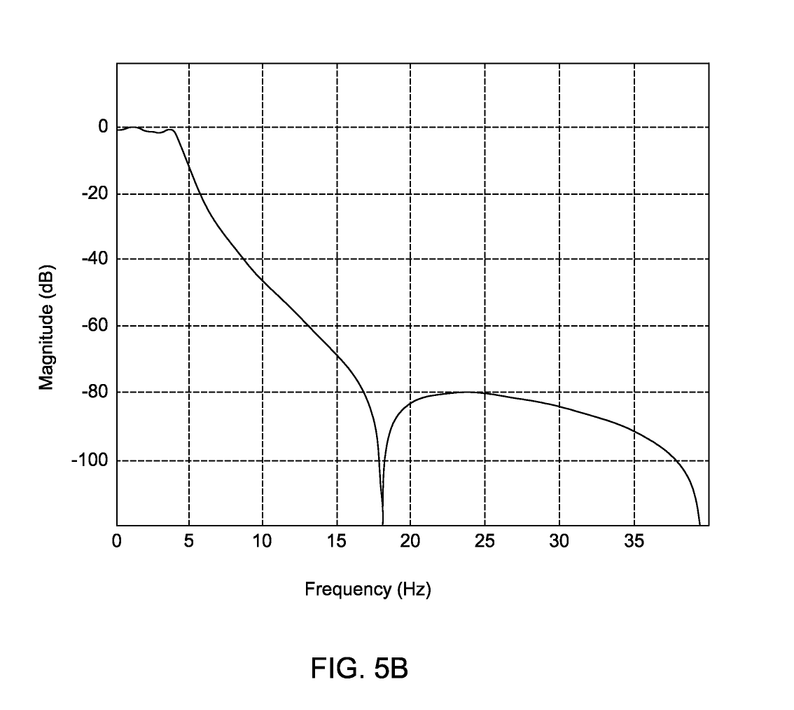

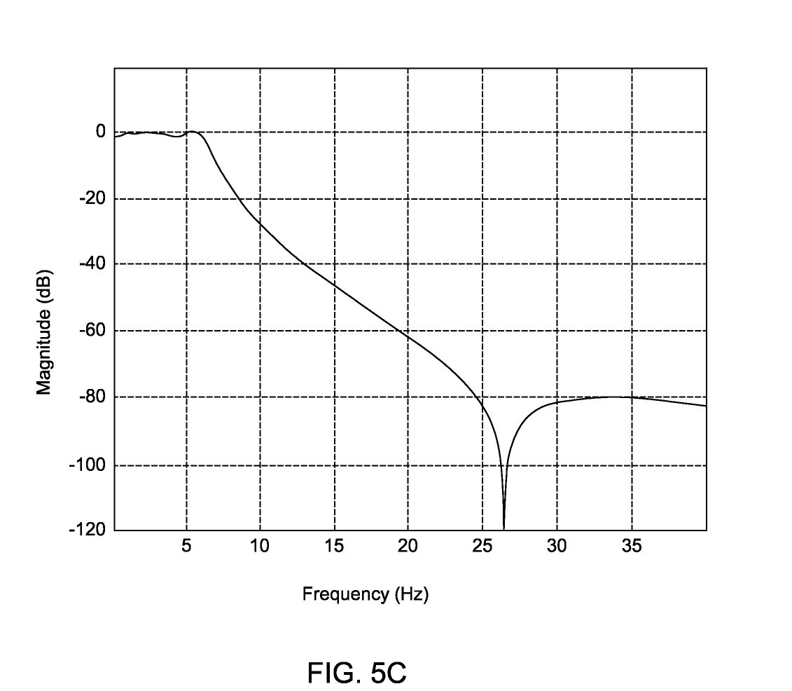

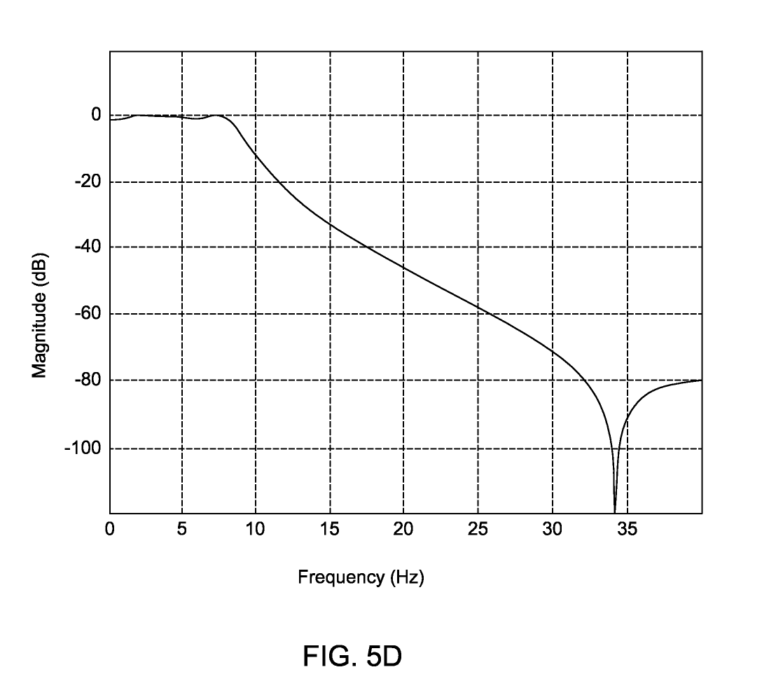

FIG. 5a illustrates a first low pass filter that includes a low pass cutoff frequency of approximately 2 Hz. The low pass filter illustrated in FIG. 5a is defined by digital filtering coefficients that are stored in the memory unit 50 shown in FIG. 1. When the processing unit 16 determines that the low pass cutoff frequency should be 2 Hz, the filtering coefficients that create the filter shown in FIG. 5a are selected and retrieved.

FIG. 5b illustrates a second low pass filter having a low pass cutoff frequency of 4 Hz. The low pass filter shown in FIG. 5b is defined by a set of digital filter coefficients that are stored within the memory unit 50. When the processing unit 16 determines that the low cutoff frequency should be 4 Hz, the filtering coefficients associated with the filter of FIG. 5b are retrieved from the memory unit 50.

FIG. 5c illustrates a low pass filter that includes a low pass cutoff frequency of 6 Hz. The filter shown in FIG. 5c is defined by a series of digital filter coefficients that are stored within the memory unit 50. When the processing unit 16 determines that the low pass cutoff frequency should be 6 Hz, the processing unit 16 retrieves the filter coefficients associated with the filter of FIG. 5c.

FIG. 5d illustrates a low pass filter that includes a low pass cutoff frequency of 8 Hz. The low pass filter shown in FIG. 5d is defined by a set of digital filter coefficients that are stored within the memory unit 50. When the processing unit 16 determines that the low pass cutoff frequency should be 8 Hz, the processing unit 16 retrieves the filter coefficients associated with the filter shown in FIG. 5d.

The low pass filters shown in FIGS. 5a-5d are fourth order elliptical filters. However, it should be understood that the order of the filter selected, the sampling rate and other known factors influence the type of low pass filter that could be used in accordance with the present disclosure. Typically, the low pass filter coefficients will be picked to keep the highest desired harmonic just below the low pass cutoff frequency in order to optimally remove any artifact and any higher harmonic energy in a consistent manner.

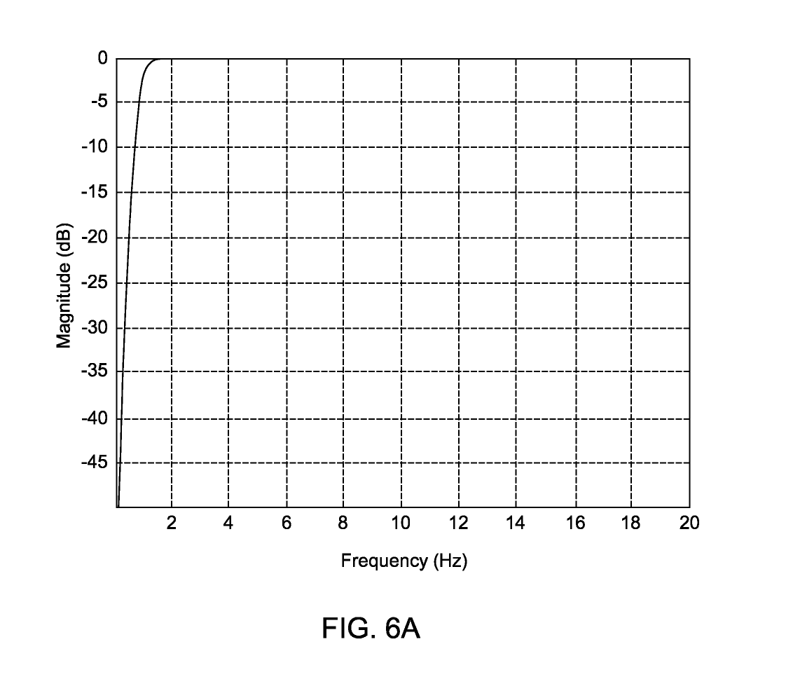

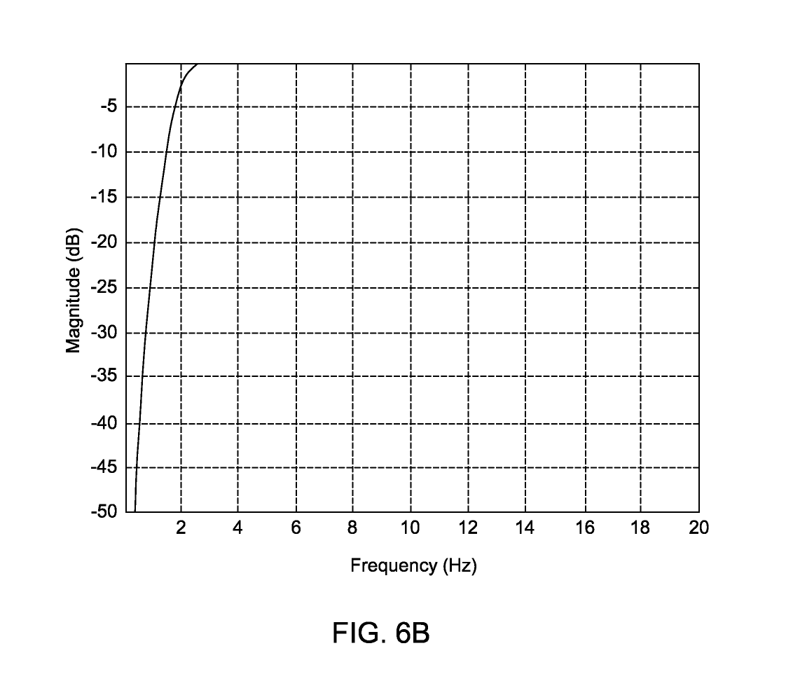

FIG. 6a illustrates a high pass filter that includes a high pass cutoff frequency of 1 Hz. The high pass filter shown in FIG. 6a is defined by a series of digital filter coefficients that are stored within the memory unit 50. When the processing unit 16 determines that the high pass cutoff frequency should be 1 Hz, the processing unit 16 retrieves the filter coefficients that are associated with the filter shown in FIG. 6a.

FIG. 6b illustrates a high pass filter that includes a high pass cutoff frequency of 2 Hz. The high pass filter shown in FIG. 6b is defined by a series of digital filter coefficients that are stored within the memory unit 50. When the processing unit 16 determines that the high pass cutoff frequency should be 2 Hz, the processing unit 16 retrieves the filter coefficients associated with the high pass filter shown in FIG. 6b.

The high pass filters shown in FIGS. 6a-6b are fourth order Butterworth filters. However, it should be understood that the order of the filter selected, the sampling rate and other known factors influence the type of high pass filters that could be used in accordance with the present disclosure. Typically the high pass filter coefficients are chosen to keep the fundamental frequency just above the high pass cutoff frequency in order to optimally remove any lower frequency artifact.

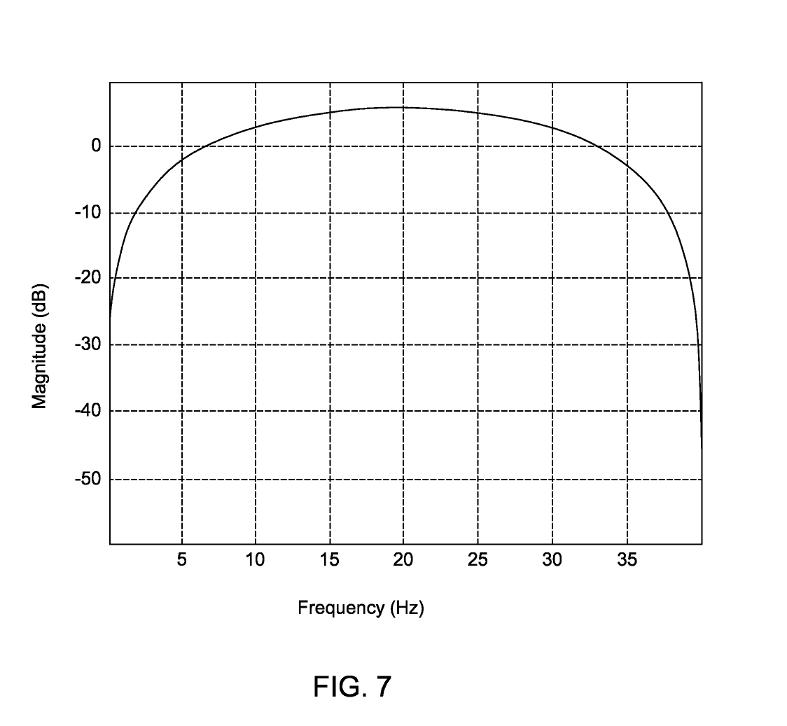

FIG. 7 illustrates another type of high pass filter referred to as a differentiator. The sixth order differentiator shown in FIG. 7 also is defined by a set of digital filter coefficients and can be used as a high pass filter having a defined high pass cutoff frequency. When the processing unit 16 determines that the high pass cutoff frequency should be as shown in FIG. 7, the processing unit 16 retrieves the filtering coefficient stored in the memory unit 50 associated with the filter of FIG. 7.

Referring back to FIG. 3, once the processing unit 16 chooses both the high pass and low pass filter coefficients in step 58, the processing unit receives a cuff pressure waveform in the time domain from the pressure transducer, as illustrated in step 60. The cuff pressure waveform acquired in step 60 is received at the processing unit 16 and the cuff pressure waveform is processed in the time domain in step 62 utilizing the pressure waveform filter or filters selected in step 58.

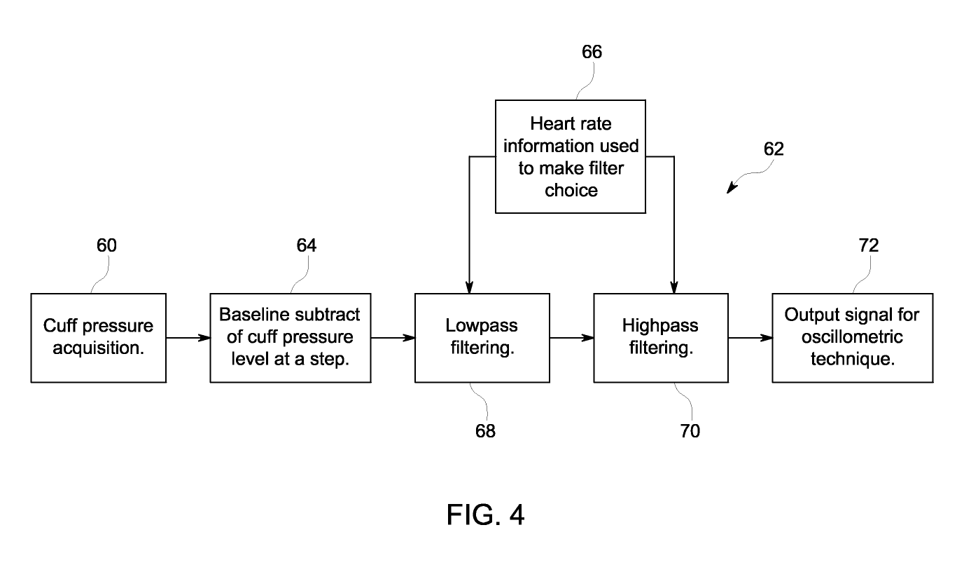

The pressure waveform processing identified by step 62 of FIG. 3 is further described in the flow diagram of FIG. 4. As illustrated in FIG. 4, the cuff pressure waveform is acquired in step 60 and the cuff pressure at the current pressure step is subtracted from the waveform as illustrated in step 64.

After the baseline pressure has been subtracted, the processing unit utilizes the heart rate information to make a filter choice, as shown in step 66 and described previously. The processing unit selects both low pass filtering coefficients in step 68 and high pass filtering coefficients in step 70. The high pass and low pass filtering coefficients selected in step 68 and 70 are retrieved from the memory unit 50 based on the desired high pass and low pass frequencies, as previously described.

Once the low pass and high pass filtering coefficients have been selected in steps 68 and 70, the processing unit initializes the filter to prevent ringing and other transient effects from dominating the filter output. The initially priming of the filter is a well-known technique. Once the filters have been primed, the pressure waveform from the blood pressure cuff is processed and an output signal is provided in step 72. The output signal provided in step 72 has been filtered to remove artifacts outside of the pass band determined by the high pass and low pass cutoff frequencies.

Referring back to FIG. 3, once the output signal has been processed in step 62, the processing unit 16 processes the pressure waveform to create oscillometric envelope data in step 74 utilizing known techniques. The oscillometric envelope data generated in step 74 is used to calculate a blood pressure estimation in step 76. As previously described, the blood pressure estimation output in step 76 includes an estimate of the systolic, mean arterial pressure and diastolic pressure for the patient.

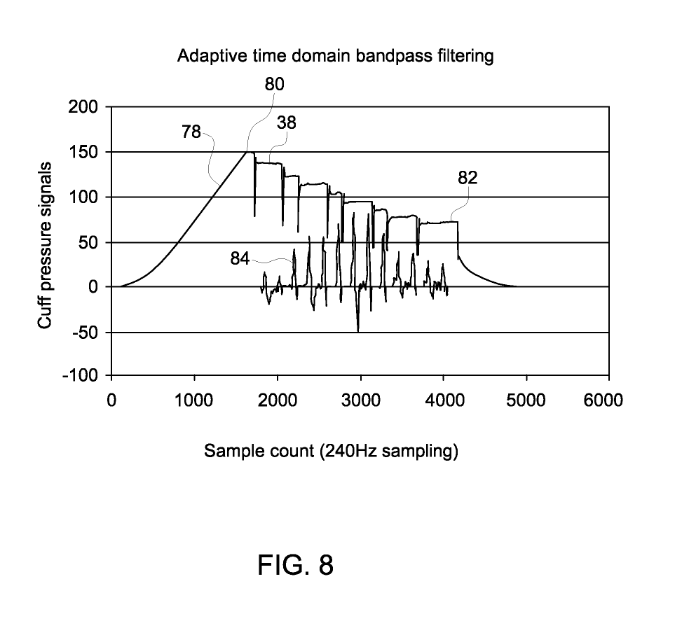

FIG. 8 illustrates the blood pressure cuff pressure 78 over the series of pressure steps 38 required to reduce the pressure of the cuff from the initial inflation pressure 80 to a final cuff pressure 82. FIG. 8 also illustrates the filtered cuff pressure waveform 84 obtained from the blood pressure cuff and filtered as previously described. The filtered cuff pressure 84 has had a significant amount of the artifacts removed for further processing by the NIBP monitoring system 10 using the techniques described in the present disclosure.

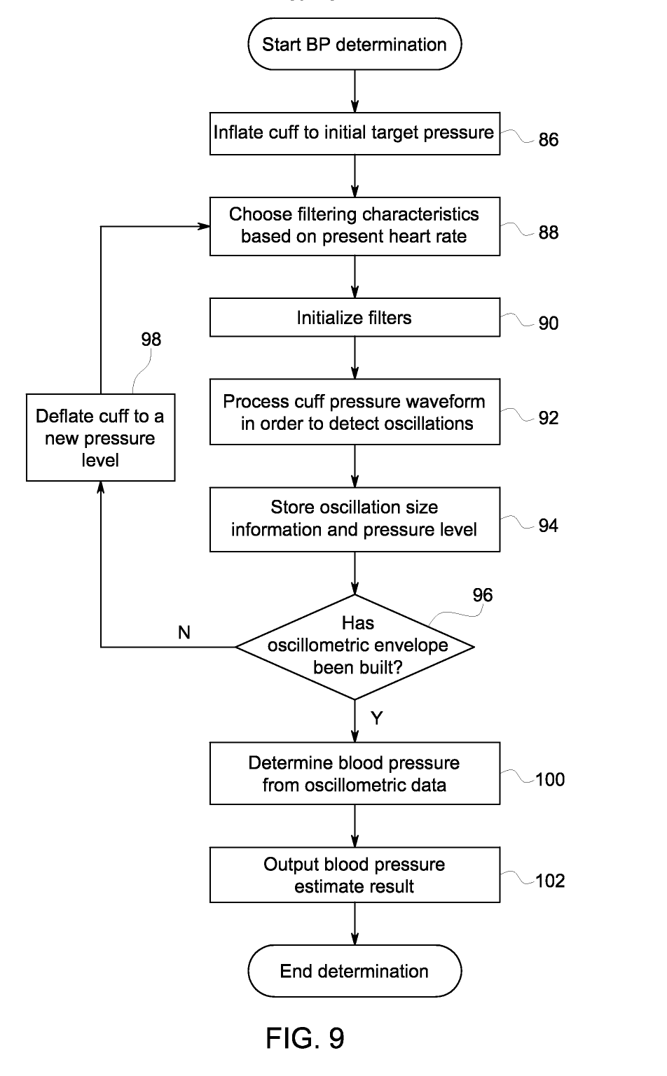

Referring now to FIG. 9, thereshown is a flowchart of the steps carried out by the processing unit in determining the blood pressure of a patient utilizing the NIBP monitoring system of the present disclosure. Initially, the processing unit 16 issues a command to the inflate valve 18 to inflate the blood pressure cuff 12 to the initial target pressure, as illustrated by step 86 of FIG. 9. Once the system reaches the initial inflation pressure 38a shown in FIG. 2, the system then determines the heart rate of the patient from the heart rate monitor 32. Based upon the heart rate information, the system chooses filtering characteristics based upon the determined heart rate, as shown in step 88. As previously described, in an embodiment in which the filtering includes both the fundamental frequency and the first and second harmonics, the high and low pass cutoff frequencies are determined and the processing unit 16 retrieves corresponding filtering coefficients for these cutoff frequencies from the memory unit 50.

Once the filtering coefficients have been selected, the system initializes the filters in step 90. After the filters have been initialized, the processing unit receives the cuff pressure signal from the pressure transducer 26 and processes the cuff pressure signal to remove artifacts outside of the pass band and detect oscillations in step 92. As shown in FIG. 8, the oscillations are present at each of the pressure steps and are relatively artifact-free based upon the adaptive filtering.

Once the oscillation amplitudes have been identified, the processing unit 16 stores the oscillation amplitudes and the pressure level of the cuff, as illustrated in step 94. After each of the oscillation amplitudes are stored in step 94, the system then determines in step 96 whether the entire oscillometric envelope has been built, as illustrated in step 96. If the entire oscillometric envelope has not yet been built, the system deflates the blood pressure cuff to a new pressure level in step 98. As illustrated in FIG. 2, the pressure of the blood pressure cuff is deflated in a series of pressure steps 38 from the initial inflation pressure 38a to a final pressure 38u.

After the cuff pressure has been deflated to a new pressure step, the system returns to step 88 and again chooses the filtering characteristics based upon the present heart rate. In this manner, the system checks the heart rate of the patient at each of the individual pressure steps such that if the heart rate changes during the blood pressure monitoring, the system may select different filter settings based upon the currently determined heart rate. Therefore, the system adapts to a changing heart rate during the process of determining the blood pressure.

The system continues to repeat steps 88-96 until the processing unit determines that the oscillometric envelope has been built in step 96. Once the oscillometric envelope has been built, the system determines the blood pressure from the oscillometric data in step 100. The determination of the blood pressure from the oscillometric data is a well-known processing technique.

Once the blood pressure oscillometric data has been fully obtained using the adaptive filler waveform output in step 100, the processing unit determines the blood pressure estimate in step 102, also in a convention& manner.

As described above, the system and method of the present disclosure selects various filtering coefficients for processing oscillometric data from a blood pressure cuff in a time domain based upon the heart rate of the patient. As the heart rate of the patient changes, the system and method of the present disclosure adjusts the filtering coefficients such that the filtering coefficients are most properly selected based upon the current heart rate of the patient. The filtering characteristics are determined at each pressure step as the pressure of the blood pressure cuff decreases from the initial inflation pressure to a final pressure. Therefore, the system and method of the present disclosure modifies the filtering coefficients during the process of determining the blood pressure of the patient. This adaptive time domain filtering technique and system enhances the removal of artifacts prior to the determination of the blood pressure estimate.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of computing a blood pressure of a patient comprising the steps of:
receiving a cuff pressure waveform in a processing unit from a blood pressure cuff positioned on the patient;
receiving an indication of the heart rate of the patient in the processing unit;
selecting filtering parameters based on the heart rate of the patient;
filtering the cuff pressure waveform in the processing unit based on the selected filtering parameters; and
determining the blood pressure of the patient in the processing unit based on the filtered cuff pressure waveform.

2. The method of claim 1 wherein the heart rate indication is received from an ECG signal from the patient.

3. The method of claim 1 wherein the heart rate indicator is received from an SpO₂ signal from the patient.

4. The method of claim 1 wherein the step of selecting filtering parameters includes:
calculating the fundamental frequency of the heart rate;
selecting a high pass cutoff frequency based on the fundamental frequency; and
selecting a low pass cutoff frequency based on a selected harmonic frequency of the fundamental frequency.

5. The method of claim 4 wherein the selected harmonic frequency is the second harmonic frequency.

6. The method of claim 5 wherein the cuff pressure waveform is processed using the selected high pass and low pass cutoff frequencies.

7. The method of claim 1 further comprising the steps of:
deflating the blood pressure cuff in a series of pressure steps from an initial inflation pressure;
receiving the cuff pressure waveform at each of the pressure steps;
filtering the cuff pressure waveform at each of the pressure steps using the selected filtering parameters; and
creating an oscillometric envelope based upon the filtered cuff pressure waveform.

8. The method of claim 4 further comprising the steps of:
retrieving a coefficient set from a memory unit based on the selected high pass and low pass cutoff frequencies; and
filtering the cuff pressure waveform based upon the retrieved coefficients.

9. A method of processing a cuff pressure waveform received from a blood pressure cuff positioned on a patient, the method comprising the steps of:
receiving an indication of the heart rate of the patient in a processing unit;
selecting filtering parameters for filtering the cuff pressure waveform based upon the heart rate of the patient; and
filtering the cuff pressure waveform in the processing unit based upon the selected filtering parameters.

10. The method of claim 9 wherein the heart rate indicator is received from an ECG signal from the patient.

11. The method of claim 9 wherein the heart rate indication is received from an SpO₂ signal from the patient.

12. The method of claim 9 wherein the step of selecting filtering parameters includes:
calculating a fundamental frequency of the heart rate;
selecting a high pass cutoff frequency based on the fundamental frequency; and
selecting a low pass cutoff frequency based on a selected harmonic frequency of the fundamental frequency.

13. The method of claim 12 wherein the selected harmonic frequency is the second harmonic frequency.

14. The method of claim 13 wherein the cuff pressure waveform is processed using the selected high pass and low pass cutoff frequencies.

15. The method of claim 12 further comprising the steps of:
deflating the blood pressure cuff in a series of pressure steps from an initial inflation pressure;
receiving an indication of the heart rate of the patient when the blood pressure cuff is at each of the series of pressure steps;
receiving the cuff pressure waveform at each of the pressure steps;
selecting filtering parameters at each pressure step based on the heart rate at the pressure step;
filtering the cuff pressure waveform at each of the pressure steps using the selected filtering parameters; and
creating an oscillometric envelope based upon the processed cuff pressure waveform.

16. The method of claim 12 further comprising the steps of:
retrieving a coefficient set from a memory unit based on the selected high pass and low pass cutoff frequencies; and
processing the cuff pressure waveform based upon the retrieved coefficients.

17. A system for determining the blood pressure of a patient, the system comprising:
a processing unit;
a heart rate monitor connected to the patient to determine the heart rate of the patient, wherein the heart rate monitor communicates the determined heart rate to the processing unit;
a blood pressure cuff positioned on the patient to obtain a cuff pressure waveform from the patient, wherein the cuff pressure waveform is provided to the processing unit;
a memory unit in communication with the processing unit, wherein the memory unit includes a series of filtering coefficients;
a low pass filter contained in the processing unit and having a low pass cutoff frequency determined by the heart rate of the patient; and
a high pass filter contained within the processing unit and having a high pass cutoff determined by the heart rate of the patient.

18. The system of claim 17 wherein the coefficients are retrieved from the memory unit based upon the high pass cutoff frequency and the low pass frequency.

19. The system of claim 17 wherein the heart rate monitor is an ECG monitor.

20. The system of claim 17 wherein the heart rate monitor is an SpO₂ monitor.
